(19) United States
(12) Patent Application Publication
(10) Pub. No.: US 2011/0190727 A1
(43) Pub. Date: Aug. 4, 2011

(54) INTERVASCULAR CATHETER, SYSTEM AND METHOD

(75) Inventors: Kevin D. Edmunds, Ham Lake, MN (US); Roger N. Hastings, Maple Grove, MN (US); Michael J. Pikus, Golden Valley, MN (US); Eric A. Mokelke, White Bear Lake, MN (US)

(73) Assignee: Boston Scientific Scimed, Inc., Maple Grove, MN (US)

(21) Appl. No.: 13/019,830

(22) Filed: Feb. 2, 2011

Related U.S. Application Data
(60) Provisional application No. 61/300,605, filed on Feb. 2, 2010.

(51) Int. Cl. A61M 25/10; B23P 17/04

(52) U.S. Cl. 604/509; 604/99.02; 29/428

(57) ABSTRACT

The present disclosure relates to medical devices and methods, for use in a vascular system. The apparatus includes a balloon catheter having an elongate body, balloon, and fluid control valve for ischemic post-conditioning therapy and/or reperfusion.

### PRIORITY INFORMATION

This application claims priority to U.S. Provisional Application No. 61/300,605 filed on Feb. 2, 2010, the specification of which is incorporated herein by reference.

### TECHNICAL FIELD

The present disclosure relates generally to medical devices and methods; and more particularly to a balloon catheter, and methods of using and forming the balloon catheter.

### BACKGROUND

Ischemia describes a situation in which the flow of oxygenated blood is restricted to a bodily organ, tissue, or part caused by constriction or obstruction of the fluid vessels. Cardiac ischemia occurs when the fluid flow to the myocardium is decreased by a partial or complete blockage of a coronary artery.

Patients who have suffered from acute myocardial infarction due to blockage of a coronary artery are susceptible to irreversible tissue damage to the heart if the myocardium is deprived of adequate levels of oxygenated fluid for a prolonged interval of time. Cardiac ischemia may also cause a serious abnormal heart rhythm, which can cause fainting and/or even sudden death.

Treatments for myocardial infarction include balloon angioplasty or bypass surgery to clear the blockage in the coronary arteries, thereby restoring the flow of oxygenated fluid flow to the heart. After a patient has suffered acute myocardial infarction, the doctor's treatment can include unblocking the clogged arteries so that reperfusion of the heart muscle can begin. Reperfusion is the restoration of the fluid flow to an organ or tissue that has had the fluid supply interrupted. Early reperfusion minimizes the extent of heart muscle damage and preserves the pumping function of the heart. However, reperfusion also has the potential to introduce additional injury. Reperfusion injury refers to the damage to tissue caused when the oxygenated fluid supply returns to the tissue after a period of ischemia.

Ischemic post-conditioning therapy is a treatment used to prevent injury after a period of ischemia occurs or help decrease the risk of a patient experiencing reperfusion injury while undergoing reperfusion. Ischemic post-conditioning therapy exposes the myocardial tissue to several repeated cycles of reperfusion and ischemia. The repeated cycles of reperfusion and ischemia is believed to help better protect cardiac tissue from the harmful effects of more prolonged episodes of ischemia or reperfusion.

### BRIEF DESCRIPTION OF THE DRAWINGS

FIG. 1 illustrates an example of a balloon catheter according to the present disclosure.

FIG. 2 illustrates an example of a balloon catheter according to the present disclosure.

FIG. 3 illustrates an example of a balloon catheter according to the present disclosure.

FIG. 4A illustrates an example of a balloon catheter according to the present disclosure.

FIG. 4B illustrates a cross-sectional view of the balloon catheter illustrated in FIG. 4A taken along lines 4B-4B.

FIG. 5 illustrates a cross-sectional view of another example of a balloon catheter illustrated in FIG. 4A taken along lines 4B-4B.

FIG. 6A illustrates an example of a balloon catheter according to the present disclosure.

FIG. 6B illustrates a cross-sectional view of the balloon catheter illustrated in FIG. 6A taken along lines 6B-6B.

FIG. 7A illustrates an example of a balloon catheter according to the present disclosure.

FIG. 7B illustrates a cross-sectional view of the balloon catheter illustrated in FIG. 7A taken along lines 7B-7B.

FIG. 7C illustrates a cross-sectional view of the balloon catheter illustrated in FIG. 7A taken along lines 7C-7C.

FIG. 7D illustrates a cross-sectional view of the balloon catheter illustrated in FIG. 7A taken along lines 7D-7D.

### DETAILED DESCRIPTION

Embodiments of the present disclosure are directed to balloon catheters, methods of their use and methods of their manufacture. For the various embodiments, the balloon catheter includes a fluid control valve that can be used to regulate a flow of fluid at an adjustable rate past the balloon catheter in its expanded state. For the various embodiments, the fluid control valve of the balloon catheter may be useful in helping to treat an organ and/or tissue of the body (e.g., heart, kidney, brain, muscle, among others) that is to undergo and/or that has undergone an ischemic event. According to the present disclosure, the balloon catheters and methods of their use can be for, among other things, ischemic post-conditioning therapy and/or reperfusion therapy, as discussed herein.

Reperfusion therapy is a term used to describe the act of reestablishing blood flow and oxygen supply to tissue that has undergone an ischemic event. Reperfusion can help the survival of cells within an ischemic area. The absence of oxygen and nutrients from blood may create a condition in which the restoration of circulation results in inflammation and oxidative damage through the induction of oxidative stress rather than restoration of normal function. Timely reperfusion limits infarct size and early reperfusion provides benefits of reduced damage.

Ischemic conditioning therapy is a procedure to protect a bodily organ, tissue, or part of the body from the damage of infarction. The procedure may consist of one or more short episodes of ischemia and reperfusion before and/or after a sustained ischemia event which may protect the organ and/or tissue against infarction and reduce the final infarct size. Ischemic conditioning therapy can include post-conditioning therapy and/or pre-conditioning therapy. Ischemic post-conditioning therapy consists of brief cycles of ischemia and reperfusion given after a prolonged ischemia event and at the onset of reperfusion. On the other hand, pre-conditioning therapy consists of administering the brief cycles of ischemia and reperfusion before a prolonged ischemia event and reperfusion are about to occur. Both post-conditioning and pre-conditioning therapy may help protect bodily tissue, organs, or other parts from ischemic-reperfusion damage.

Various embodiments of the present disclosure are illustrated in the figures. As will be appreciated, elements of the various embodiments illustrated in the figures and discussed herein can be added, exchanged, and/or eliminated so as to provide a number of additional embodiments of the balloon catheter of the present disclosure. In addition, as will be appreciated the proportion and the relative scale of the elements provided in the figures are intended to illustrate the embodiments of the present disclosure, and should not be taken in a limiting sense.

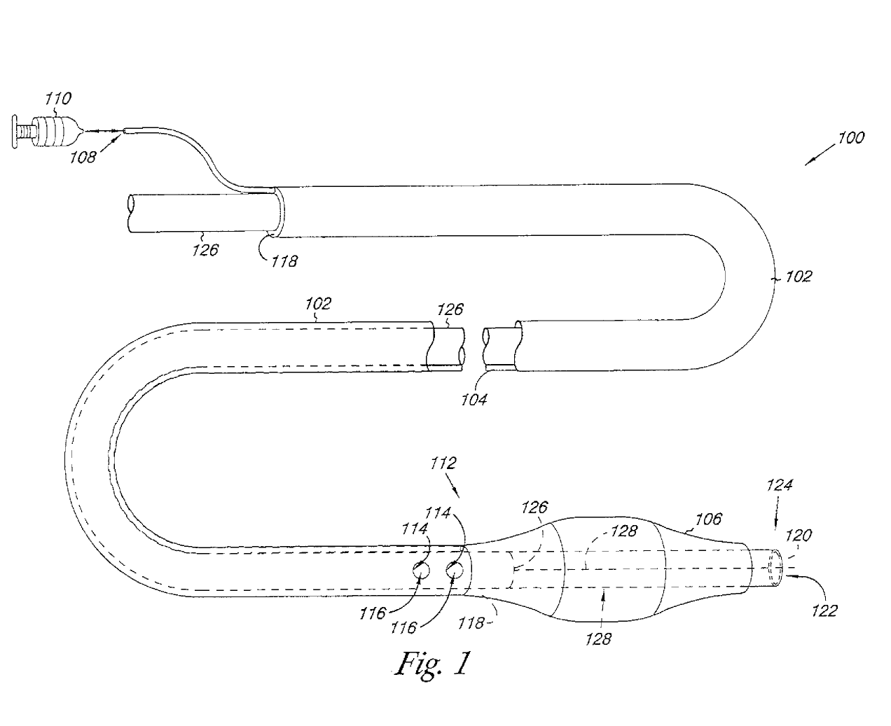

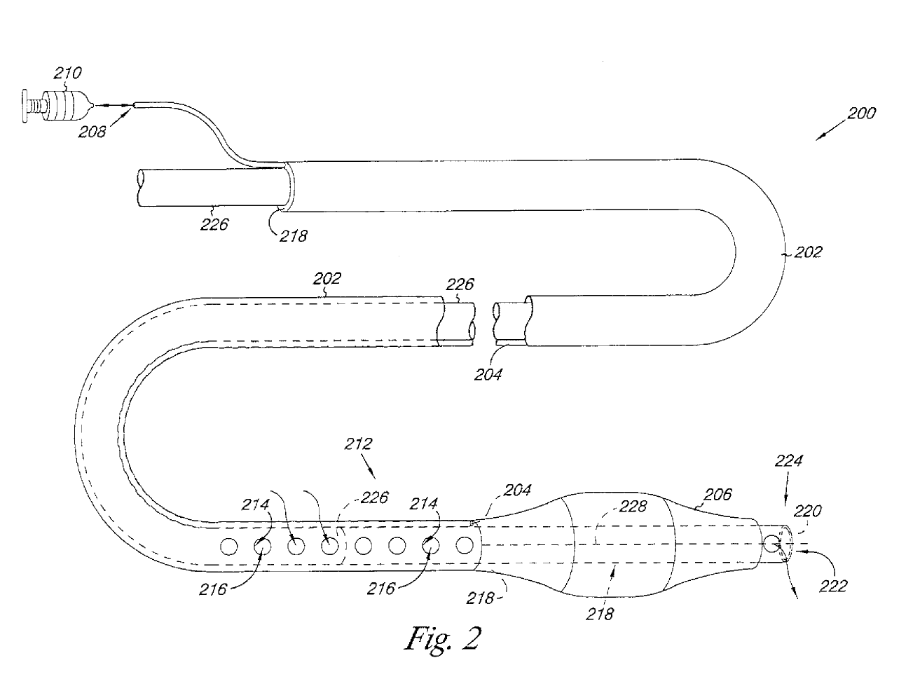

The figures herein follow a numbering convention in which the first digit or digits correspond to the drawing figure number and the remaining digits identify an element or component in the drawing. Similar elements or components between different figures may be identified by the use of similar digits. For example, 110 may reference element “10” in FIG. 1, and a similar element may be referenced as 210 in FIG. 2. The features of the drawing are not to scale.

FIG. 1 provides an embodiment of a balloon catheter 100 of the present disclosure. The balloon catheter 100 includes an elongate body 102 having an inflation lumen 104 that extends through the elongate body 102. The balloon catheter 100 also includes a balloon 106 coupled to the elongate body 102 and in fluid communication with the inflation lumen 104. For the various embodiments, the inflation lumen 104 couples an inflation port 108 to an expandable volume defined at least in part by the balloon 106 and the elongate body 102 of the balloon catheter 100.

For the various embodiments, an inflation device 110 can be releasably coupled to the balloon catheter 100 (e.g., to the inflation port 108) from which a fluid (e.g., saline) can be transferred under pressure to change the inflation state (e.g., an expanded state and an un-expanded state) of the balloon 106. FIG. 1 illustrates an embodiment in which the balloon 106 is in its expanded state.

The balloon catheter 100 further includes a fluid control valve 112 integrated with the balloon catheter 100. For the various embodiments, the fluid control valve 112 can regulate a flow of a fluid (e.g., blood) at an adjustable rate past the balloon 106 while it is in an expanded state. For the various embodiments, the expanded state of the balloon 106 includes a state in which an exterior diameter of the balloon 106 no longer increases from adding additional fluid pressure from the inflation device 110.

For the various embodiments, the fluid control valve 112 can regulate the flow of the fluid past the balloon 106 in an expanded state at the adjustable rate that is greater than a no-flow rate and less than a full-flow rate of the fluid control valve 112. In other words, the fluid control valve 112 can regulate the flow of fluid past the balloon 106 in its inflated state at a flow rate between the no-flow rate and the full-flow rate of the fluid control valve 112.

For the various embodiments, the no-flow rate can occur when both the balloon 106, in its expanded state, and the fluid control valve 112, in a fully closed state, block or prevent the flow of the fluid past the balloon 106. In other words, there is no fluid flow through the fluid control valve 112 and/or around the exterior surface(s) of the balloon 106 in its expanded state. For the various embodiments, the full-flow rate can occur when the fluid control valve 112 is in a fully open state (e.g., can be opened no further so as to increase the flow of fluid through the fluid control valve 112 due to changing the state of the fluid control valve 112), while the balloon 106, in its expanded state, prevents the flow of fluid past its exterior surface(s). For the various embodiments, preventing flow of fluid past the exterior surface(s) of the balloon 106 can occur as a result of the balloon 106 in its inflated state being seated against a surface of a lumen in which the balloon 106 has been positioned.

For the various embodiments, the adjustable rate between the no-flow rate and the full-flow rate, as discussed herein, can be achieved by adjusting the fluid control valve 112 so as to provide an intermediate flow rate between the no-flow rate and the full-flow rate. For the various embodiments, the intermediate flow rate can be regulated and maintained for a predetermined length of time as desired by the operator of the balloon catheter 100. In other words, the fluid control valve 106 can be adjusted and held in one position so as to provide the intermediate flow rate for a sustained interval of time. Suitable examples of the sustained interval of time include, but are not limited to, as little as thirty (30) seconds to as long as five (5) minutes.

For the various embodiments, the fluid control valve 112 can include a first surface 114 defining an inlet opening 116 through the elongate body 102. As illustrated in FIG. 1, more than one of the inlet opening 116 is possible. For the various embodiments, the inlet opening 116 connects to a perfusion lumen 118 that extends longitudinally past the balloon 106 through the elongate body 102 to a second surface 120 defining an outlet opening 122 through the elongate body 102. For the various embodiments, the outlet opening 122 of the perfusion lumen 118 can be at a distal end 124 of the elongate body 102.

For the various embodiments, the fluid control valve 112 further includes a valve member 126. For the various embodiments, the valve member 126 can be used to regulate the flow of the fluid past the balloon 106 in its expanded state at the adjustable rate greater than the no-flow rate and less than the full-flow rate. For the various embodiments, the valve member 126 can be moved relative the inlet opening 116 to regulate the flow of the fluid through the fluid control valve 112. For example, the valve member 126 can move longitudinally along a longitudinal axis 128 of the elongate body 102 within the perfusion lumen 118 to regulate the flow of the fluid through the fluid control valve 112. So, for example, the valve member 126 can be positioned within the perfusion lumen 118 so that one or more of the inlet openings 116 are at least partially unobstructed by the valve member 126.

For the various embodiments, it is also possible that the valve member 126 can be positioned so as to provide the full-flow rate of the fluid though the fluid control valve 112 when none of the inlet opening 116 are obstructed with the valve member 126. For the various embodiments, it is also possible that the valve member 126 can be positioned so as to provide the no-flow rate of the fluid through the fluid control valve 112 when all of the inlet openings 116 are obstructed with the valve member 126.

For the various embodiments, the valve member 126 can also be used as a guide wire for the balloon catheter 100. For example, the valve member 126 can be advanced to a desired location within a body. The valve member 126 can then be positioned within the perfusion lumen 118 (e.g., starting at the distal end 124 of the elongate body 102) and the balloon catheter 100 advanced along the valve member 126 to position the balloon 106 at a desired location within the body. For the various embodiments, it is also possible that a guide wire lumen separate from the perfusion lumen 118 can be provided in the elongate body 102. For the various embodiments, such guide wire lumens can include, but are not limited to, rapid-exchange type lumens and full guide wire lumen designs. For the various embodiments, it is also possible to have the perfusion lumen 118 and the inflation lumen 104 in a concentric configuration, as compared to the eccentric configuration illustrated in FIG. 1.

For the various embodiments, the elongate body 102 can be formed from a suitable material, for example, but not limited to, polyoxymethylene (POM), polybutylene terephthalate (PBT), polyether block ester, polyether block amide (PEBA), fluorinated ethylene propylene (FEP), polyethylene (PE), polypropylene (PP), polyvinylchloride (PVC), polyurethane, polytetrafluoroethylene (PTFE), polyether-ether ketone (PEEK), polyimide, polyamide, polyphenylene sulfide (PPS), polyphenylene oxide (PPO), polysufone, nylon, perfluoro (propyl vinyl ether) (PFA), polyether-ester, polymer/metal composites, or mixtures, blends, or combinations thereof.

For the various embodiments, the balloon 106 can be formed from a suitable balloon material. For example, the balloon 106 can be formed from a semi-compliant material, for example, ethylene-vinyl acetate, polyvinyl chloride (PVC), olefin copolymers or homopolymers, polyethylenes, polyurethanes, crosslinked low density polyethylenes (PETS), highly irradiated linear low density polyethylene (LDPE), acrylonitrile polymers and copolymers, acrylonitrile blends, and ionomer resins, among other semi-compliant materials. For the various embodiments, the balloon 106 can also be formed from a non-compliant material, for example, polyethylene terephthalates, polyacrylenesulfide, and copolyesters, among other non-compliant materials. For the various embodiments, the balloon 106 can also be formed from a compliant material, for example, nylon, and polyamines, among other compliant materials. Other balloon materials may also be used.

For the various embodiments, the balloon catheter 100 can also include one or more areas, bands, coatings, and/or members that are detectable by imaging modalities such as X-Ray, MRI, and/or ultrasound, among others. In some embodiments at least a portion of the balloon catheter 100 is at least partially radiopaque.

FIG. 2 provides an additional embodiment of the balloon catheter 200 according to the present disclosure. As illustrated, the balloon catheter 200 includes the elongate body 202, the balloon 206 coupled to the elongate body 202 and in fluid communication with the inflation lumen 204. The balloon catheter 200 also includes the inflation port 208 and inflation device 210, as discussed herein, through which fluid can be transported to change the inflation state of the balloon 206. FIG. 2 illustrates an embodiment in which the balloon 206 is in its expanded state.

For the various embodiments, the fluid control valve 212 integrated with the balloon catheter 200 can regulate the flow of the fluid past the balloon 206 in an expanded state at the adjustable rate greater than the no-flow rate and less than the full-flow rate, as discussed herein. As illustrated, the fluid control valve 212 includes the first surface 214 defining one or more inlet openings 216 through the elongate body 202. For the various embodiments, the inlet openings 216 connect to the perfusion lumen 218 that extends longitudinally past the balloon 206 through the elongate body 202 to the second surface 220 defining the outlet opening 222 through the elongate body 202. For the various embodiments, the outlet opening 222 of the perfusion lumen 214 can include openings through the wall forming elongate body 202 in addition to the distal end 224 of the elongate body 202.

FIG. 2 provides an embodiment in which the valve member 226 is used to regulate the flow of the fluid past the balloon 206 in its expanded state at the adjustable rate greater than the no-flow rate and less than the full-flow rate. As illustrated, the valve member 226 is positioned relative the inlet opening 216 so that one or more of the inlet opening 216 are at least partially unobstructed, while the remaining inlet openings 216 are obstructed by the valve member 226.

FIG. 3 provides an additional embodiment of the balloon catheter 300 according to the present disclosure. For the various embodiments, and as illustrated in FIG. 3, the fluid control valve 312 includes a valve member 326 that has a profile that allows for the flow of the fluid through the inlet openings 316 and past the balloon 306 at the adjustable rate greater than the no-flow rate and less than the full-flow rate. As illustrated in FIG. 3, the valve member 326 can have a profile that tapers from a first diameter 330 along the length of the valve member 326 to a second diameter 332 smaller than the first diameter 330 at an end 334 of the valve member 326.

For the various embodiments, the taper of the valve member 326 can allow for regulating the flow of fluid past the balloon catheter 300 in its expanded state at the adjustable rate, as discussed herein. For example, the valve member 326 can be moved longitudinally relative the inlet openings 316 to allow for an adjustable flow rate that is great than the no-flow rate and less than the full-flow rate, as discussed herein. The valve member 326 may also be positioned relative the inlet openings 316 to provide for the no-flow rate. This can occur when the portion of the valve member 326 having the first diameter 330 is positioned over and obstructs fluid flow through the inlet openings 316. Similarly, the valve member 326 may also be positioned relative the inlet openings 312 to provide for the full-flow rate, when the valve member 326 is positioned so as not to obstruct fluid flow through the inlet openings 316.

For the various embodiments, the valve member 326 may have a number of different profiles, cross-sectional shapes, and/or dimensions along the length of the valve member 326. For example, the valve member 326 can have a cross-sectional shape that includes, but is not limited to, a circle, oval, square, triangle, trapezoid, polygon, parallelogram, rhombus, and other irregular shapes. Regardless of the profile, cross-sectional shape, and/or dimension along the length of the valve member 326, the valve member 326 may move relative the inlet openings 316 to regulate the flow of the fluid through the fluid control valve 312 at various intermediate flow rates that are between the no-flow rate and the full-flow rate.

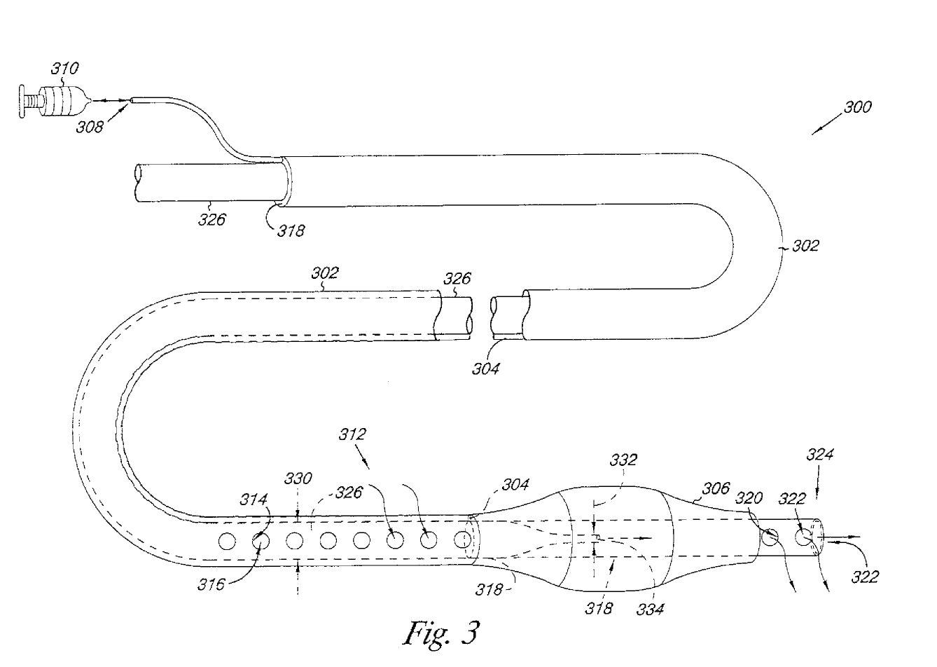

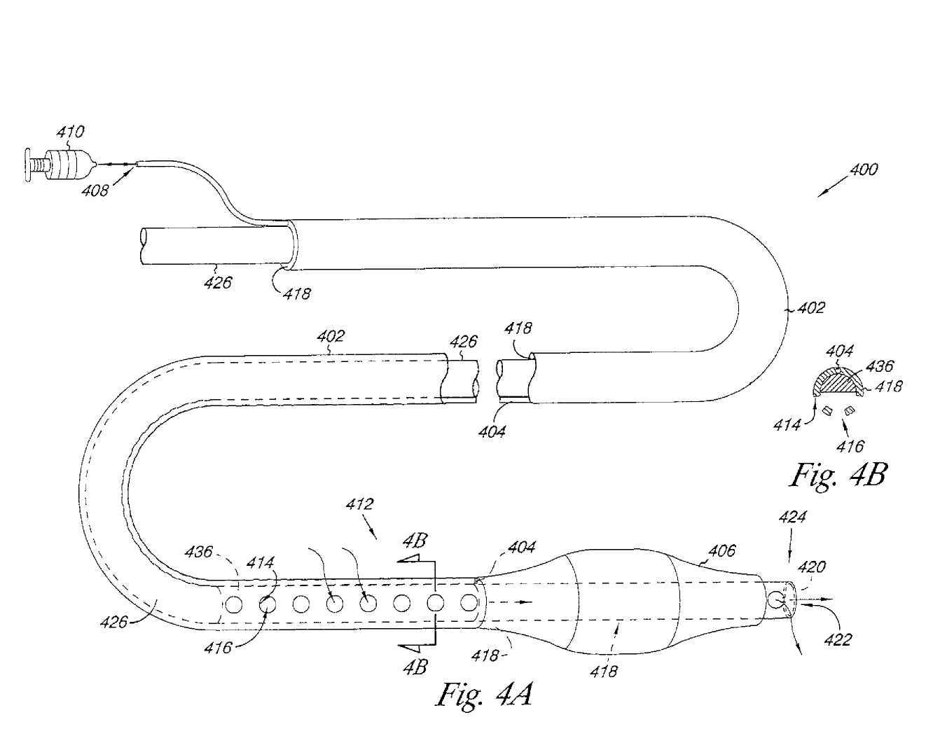

FIG. 4A provides an additional embodiment of the balloon catheter 400 according to the present disclosure. For the various embodiments, and as illustrated in FIG. 4, the valve member 426 can include a plug 436 adjacent the inlet openings 416. For the various embodiments, the valve member 426 can be used to rotate the plug 436 within the perfusion lumen 418 relative the inlet opening 416 to regulate the flow of blood past the balloon 406 at the adjustable rate greater than the no-flow rate and less than the full-flow rate, as discussed herein.

For various embodiments, the plug 436 can have a cross-sectional shape (e.g., taken across its longitudinal axis) that allows for the fluid to pass through the inlet openings 416 and into the perfusion lumen 418 when the plug 436 is in a predetermined radial position relative the inlet openings 416. FIG. 4B provides an illustration of a cross-sectional shape of the plug 436 that allows for the fluid to pass through the inlet openings 416 and into the perfusion lumen 418. As appreciated, other cross-sectional shapes are also possible. For the various embodiments, the plug 436 can be rotated within the perfusion lumen to allow for the valve member 426 to regulate the flow of the fluid past the balloon 406 at the adjustable rate greater than the no-flow rate and less than the full-flow rate.

For the various embodiments, indexing marks 438 can be provided on both the valve member 426 and the elongate body 402 to allow for the operator of the balloon catheter 400 to better know what position the plug 436 is in relative the inlet openings 416. For the various embodiments, a series of indexing marks 438 can be provided on both the elongate body 402 and the valve member 426 that indicate a no-flow rate, a full-flow rate, and intermediate flow rates between the no-flow and full-flow rates, as discussed herein.

As illustrated in FIG. 4B, the plug 436 can have a semi-circle cross-sectional shape. For the various embodiments, the plug 436 may be rotated with the valve member 426 so the portion of the semi-circle shape in contact with the inside surface of the perfusion lumen 418 can block the inlet openings 416 and prevent flow of the fluid past the balloon 406 (e.g., the no-flow rate). As the plug 436 is rotated the semi-circle shape in contact with the inside surface of the perfusion lumen 418 can be opposite the inlet openings 416, providing for the full-flow rate of the fluid control valve 412 since the inlet openings 416 are not obstructed by the semi-circle portion that is in contact with the perfusion lumen 418. Furthermore, for the various embodiments, the plug 436 may also be rotated to an intermediate position where the semi-circle obstructs some, but not all of the inlet openings 416, providing a flow rate of the fluid past the balloon 406 that is greater than the no-flow rate and less than the full-flow rate. The intermediate flow-rate may be maintained for a predetermined amount of time and may continue to be adjusted between the no-flow rate and full-flow rate by rotating the plug 436 where the semi-circle is positioned to block a portion of the inlet openings 416.

For the various embodiments, the plug 436 can have other various cross-sectional shapes. FIG. 5 provides an example of one such cross-sectional shape for the plug 536 that includes longitudinal grooves 540. In FIG. 5, the plug 536 may be rotated to position the longitudinal grooves 540 so as to provide a no-flow rate, a full-flow rate, or an intermediate rate between the no-flow rate and full-flow rate. For the various embodiments, the plug 536 in FIG. 5 may be rotated so that longitudinal grooves 540 indented into the plug 536 are positioned opposite the interior surface of the perfusion lumen 518, obstructing the inlet openings 516 and providing the no-flow rate of the fluid. Additionally, the longitudinal grooves 540 on the plug 536 may be positioned opposite the inlet openings 516 to provide a full-flow rate of the fluid past the balloon 506. Furthermore, the longitudinal grooves 540 on the plug 536 may be positioned at an intermediate position where a portion of the longitudinal grooves 540 is in fluid communication with the inlet openings 516 of the perfusion lumen 518 providing a flow rate of the fluid past the balloon that is greater than the no-flow rate and less than the full-flow rate. Other geometric shapes and/or patterns that would allow for fluid to move through the perfusion lumen 518 are also possible and the cross-sectional shape of the plug is not limited to the shapes discussed above.

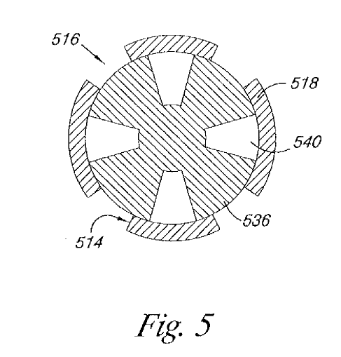

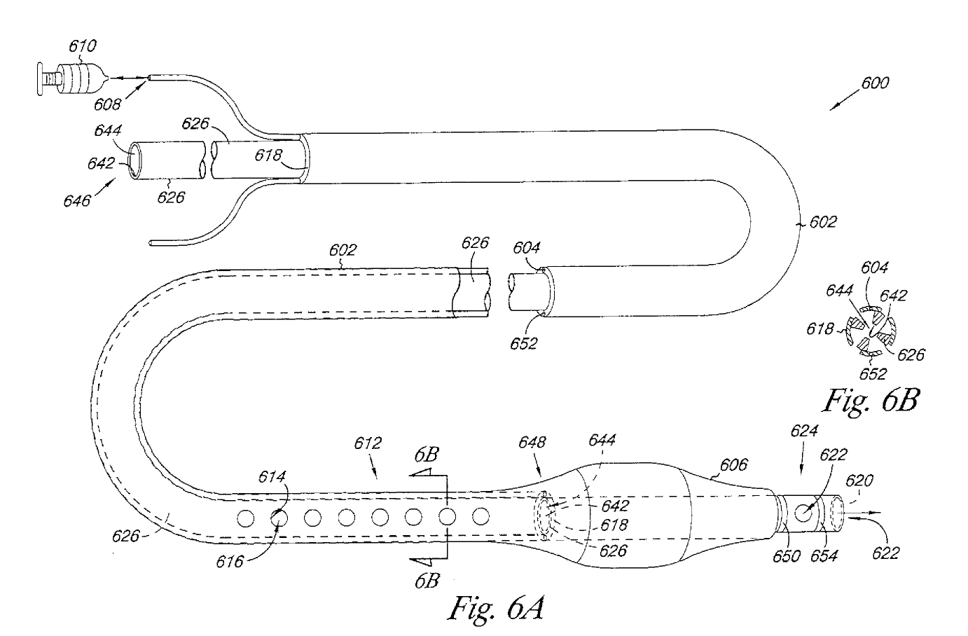

FIG. 6A illustrates an example of a balloon catheter 600 according to the present disclosure. As illustrated, the balloon catheter 600 includes the elongate body 602, the balloon 606 coupled to the elongate body 602 and in fluid communication with the inflation lumen 604. The balloon catheter 600 also includes the inflation port 608 and inflation device 610, as discussed herein, through which fluid can be transported to change the inflation state of the balloon 606. FIG. 6A illustrates an embodiment in which the balloon 606 is in its expanded state.

For the various embodiments, the fluid control valve 612 integrated with the balloon catheter 600 can regulate the flow of the fluid past the balloon 606 in an expanded state at the adjustable rate greater than the no-flow rate and less than the full-flow rate, as discussed herein. As illustrated, the fluid control valve 612 includes the first surface 614 defining one or more inlet openings 616 through the elongate body 602. For the various embodiments, the inlet openings 616 connect to the perfusion lumen 618 that extends longitudinally past the balloon 606 through the elongate body 602 to the second surface 620 defining the outlet opening 622 through the elongate body 602. For the various embodiments, the outlet opening 622 of the perfusion lumen 618 can include openings through the wall forming the elongate body 602 in addition to the distal end 624 of the elongate body 602.

FIG. 6A provides an embodiment in which the valve member 626 is used to regulate the flow of the fluid past the balloon 606 in its expanded state at the adjustable rate greater than the no-flow rate and less than the full-flow rate, as discussed herein. FIG. 6B provides an illustration of a cross-sectional shape of the valve member 626. As illustrated in FIG. 6B, the valve member 626 can be positioned relative the inlet opening 616 so that one or more of the inlet openings 616 are at least partially unobstructed, while the remaining inlet openings 616 are obstructed by the valve member 626.

FIGS. 6A and 6B further provide an embodiment in which the valve member 626 includes a surface 642 that defines a lumen 644 that extends through the valve member 626. For the various embodiments, the valve member 626 includes a first end 646 and a second end 648, where the surface 642 defining the lumen 644 extends from the first end 646 to the second end 648. For the various embodiments, the second end 648 of the lumen 644 is in fluid communication with the perfusion lumen 618. For the various embodiments, fluids (e.g., blood and/or pharmaceuticals) can be introduced and/or removed from the perfusion lumen 618 from the first end 646 of the lumen 644.

As illustrated in FIGS. 6A and 6B, the lumen 644 of the valve member 626 can be positioned within the perfusion lumen 618. Rotating the valve member 626, as discussed herein, can allow for an adjustable flow of the fluid past the balloon 606 that is greater than the no-flow rate and less than the full-flow rate by allowing a portion of the inlet openings 616 to be in fluid communication with the perfusion lumen 618.

For the various embodiments, the balloon catheter 600 can further include an electrode 650 for conducting an electrical potential. For the various embodiments, the electrode 650 can sense and/or deliver an electrical potential from and/or to the location within the body where the electrode 650 is present. For the various embodiments, the electrode 650 can be a ring electrode (e.g., an annular or semi-annular ring) having an electrically conductive lead 652 that extends through and that is electrically insulated by the elongate body 602. For the various embodiments, the electrode 650 can be used for mono-polar sensing and/or pacing. For the various embodiments, other electrodes with an electrically conductive lead may be located along the elongate body 602 to allow for bi-polar sensing and/or pacing from the balloon catheter 600.

For the various embodiments, the balloon catheter 600 can further include a flow sensor 654. For the various embodiments, the flow sensor 654 can be used to measure a flow rate of fluid through the perfusion lumen 618. For the various embodiments, the flow sensor 654 can include a pressure sensor coupled to the distal end 624 of the elongate body 602 and/or another pressure sensor positioned within the valve member 626. The pressure differential between the two pressure sensors can be compared and the flow rate of the fluid through the perfusion lumen 618 can be monitored. Examples of such an embodiment can be found in U.S. Patent Application No. 2007/0160645 entitled “Postconditioning System and Method for the Reduction of Ischemic-Reperfusion Injury in the Heart and Other Organs,” which is hereby incorporated by reference.

Additionally, the blood flow rate can be measured by thin film anemometry techniques. For the various embodiments, the flow sensor 654 can include a plurality of interconnected conductors of an alternating metal type. Junctions between the conductors of the thermopile can be located near the longitudinal end of the flow sensor. A voltage can be generated by the thermopile and the difference in temperature can be determined. The blood flow rate is measured by introducing an alternating current through the thermopile to heat the flow sensor. The voltage generated by the thermopile is then measured to obtain a sample of the magnitude and direction of the velocity of the blood flow. Examples of such an embodiment can be found in U.S. Pat. No. 5,831,159 entitled “Thermopile Flow Sensor,” which is hereby incorporated by reference in its entirety.

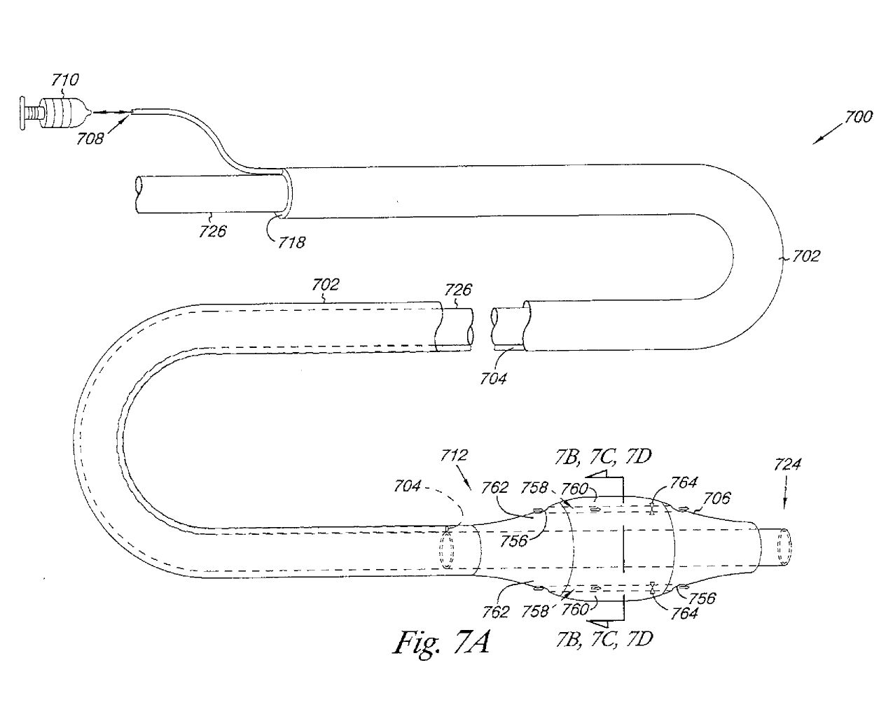

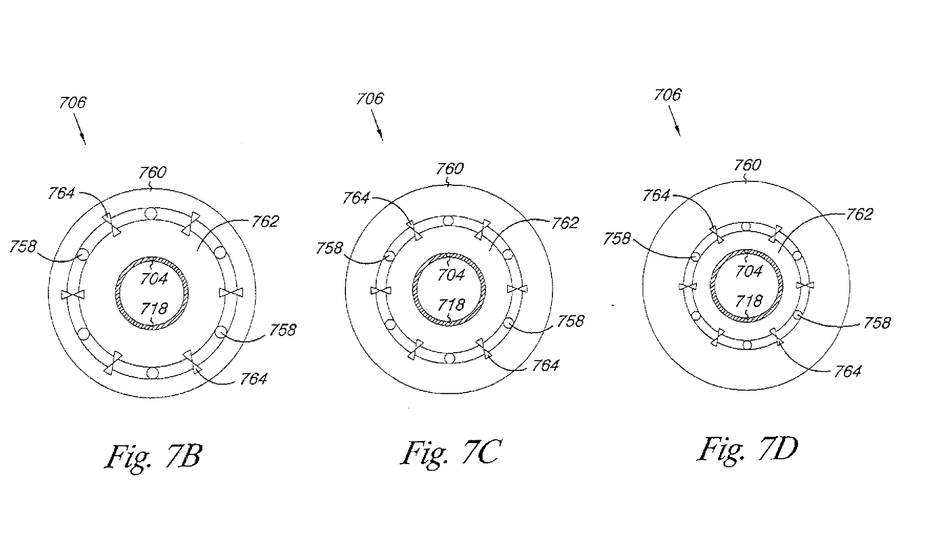

FIG. 7A illustrates an additional example of a balloon catheter 700 according to the present disclosure. As illustrated, the balloon catheter 700 includes the elongate body 702 and the balloon 706 coupled to the elongate body 702 and in fluid communication with the inflation lumen 704. The balloon catheter 700 also includes the inflation port 708 and inflation device 710, as discussed herein, through which fluid can be transported to change the inflation state of the balloon 706. FIG. 7 illustrates an embodiment in which the balloon 706 is in its expanded state.

For the various embodiments, the balloon catheter 700 includes a fluid control valve 712 integrated with the balloon 706, which can regulate the flow of the fluid past the balloon 706 in its expanded state at the adjustable rate greater than the no-flow rate and less than the full-flow rate, as discussed herein. As illustrated, the fluid control valve 712 includes a surface 756 that defines a tubular passage 758 longitudinally passing through the balloon 706 itself. For the various embodiments, changes in the inflation pressure for the balloon in its expanded state regulate the flow of the fluid through the tubular passage 758 past the balloon 706 at the adjustable rate greater than the no-flow rate and less than the full-flow rate, as discussed herein. Additionally, the balloon catheter 700 can include more than one tubular passage.

For the various embodiments, the balloon 706 and the tubular passage 758 of the fluid control valve 712 integrated therein can include an outer balloon 760 and an inner balloon 762 concentrically arranged with and in fluid communication with the outer balloon 760. For the various embodiments, the outer balloon 760 and the inner balloon 762 of the balloon 706 are in fluid communication via a port 764 that connects the interior volumes of the outer and inner balloons 760 and 762.

For the various embodiments, changes in the inflation pressure of the balloon 706 can be used to change a cross-sectional area of the tubular passage 758 so as to regulate the flow of the fluid through the tubular passage 758 and past the balloon 706. For example, at a first predetermined internal pressure value the balloon 706 can achieve its expanded state, as discussed herein. At this first predetermined internal pressure, the pressure exerted by the outer balloon 760 and the inner balloon 762 are not so great as to change the cross-sectional area of the tubular passages 758. As the internal pressure of the balloon 706 is increased past the first predetermined internal pressure value, the outer balloon 760 and the inner balloon 762 can begin to apply additional pressure to the tubular passages 758, which can then cause the tubular passage 758 to at least partially collapse. In this way, one or more of the adjustable rates greater than the no-flow rate and less than the full-flow rate, as discussed herein, can be achieved with the balloon catheter 700.

For the various embodiments, the walls of the outer balloon 760 and the inner balloon 762 that interface with the tubular passages 758 can be more compliant as compared to the walls forming the peripheral surface(s) of the balloon 706. For the various embodiments, this configuration can allow the walls of the outer balloon 760 and the inner balloon 762 that interface with the tubular passages 758 to preferentially expand (e.g., squeeze) the tubular passages 758 instead of expanding the outer perimeter of the balloon 706.

FIGS. 7B-7D illustrate cross-sectional views of the balloon catheter 700 illustrated in FIG. 7A during different flow rates, as discussed herein. For example, FIG. 7B provides an illustration of the balloon 706 in its expanded state (e.g., at the first predetermined internal pressure) that can provide a full-flow rate condition (e.g., the tubular passages 758 can have no greater diameter). FIG. 7C provides an illustration of the balloon 706 in its expanded state having an internal pressure at a second predetermined internal pressure that allows for the no-flow rate (e.g., the tubular passages 758 completely collapsed) as discussed herein. FIG. 7D provides an illustration of the balloon 706 in its expanded state having an internal pressure that is between the first predetermined internal pressure and the second predetermined internal pressure that allows for one or more of the intermediate flow rates, as discussed herein, through the tubular passages 758. As appreciated, the values for the first and second predetermined internal pressure will be a function of the configuration and materials used in forming the balloon catheter 700.

Embodiments of the present disclosure can further include methods of forming the balloon catheter of the present disclosure. For example, the methods can include forming a balloon catheter, as discussed herein. The method includes providing an elongate body having an inflation lumen. The method further includes coupling a balloon to the elongate body to be in fluid communication with the inflation lumen and integrating a fluid control valve with the balloon catheter. The fluid control valve regulates a flow of fluid (e.g., blood) past the balloon while it is in an expanded state at an adjustable rate greater than a no-flow rate and less than a full-flow rate of the fluid control valve.

For the various embodiments, integrating the fluid control valve with the balloon catheter can include providing a perfusion lumen through at least a portion of the elongate body. Integrating the fluid control valve with the balloon catheter can further include forming an inlet opening through the elongate body adjacent a first end of the balloon to connect with the perfusion lumen that extends past the balloon and forming an outlet opening for the perfusion lumen through the elongate body adjacent a second end of the balloon. Additionally, integrating the fluid control valve with the balloon catheter can include providing a valve member for the fluid control valve, where the valve member moves at least partially within the perfusion lumen and relative the inlet opening to regulate the flow of the fluid through the control valve.

For the various embodiments, integrating the fluid control valve with the balloon can include forming a lumen in the valve member, where the lumen can pass a liquid into the perfusion lumen of the elongate body. Moreover, the method can include providing an electrode on the, balloon catheter, where the electrode can conduct an electrical potential. For the various embodiments, the method can include providing an electrically conductive lead that extends through and that is electrically insulated by the elongate body.

Additional embodiments of integrating the fluid control valve with the balloon catheter can include forming a tubular passage longitudinally past the balloon, where inflation pressure of the balloon regulates a flow of the fluid past the balloon in an expanded state at an adjustable rate greater than a no-flow rate and less than a full-flow rate of the fluid control valve. Furthermore, for the various embodiments, forming a tubular passage can include forming an outer balloon and an inner balloon concentrically arranged with and in fluid communication with the outer balloon. The method can further include positioning the tubular passage past the balloon between the outer balloon and the inner balloon so that changes in inflation pressure of the balloon change a cross-sectional area of the tubular passage to regulate the flow of the fluid past the balloon.

The embodiments of the catheter described herein may be used to provide ischemic post-conditioning therapy and/or reperfusion to organs and/or tissue in the body. For example, the apparatus can include a balloon catheter that can be used to restore the fluid flow to an organ and/or tissue (e.g., heart, kidney, brain, muscle, among others) that has suffered ischemia. For the various embodiments of the present disclosure the balloon catheter may help to condition the organ and/or tissue for a subsequent ischemic event or reperfusion.

In addition, a method includes positioning the balloon of the balloon catheter, as discussed herein, at a predetermined vascular location of a patient using minimally invasive percutaneous, transluminal techniques. For example, a guidewire can be positioned within the vascular system of a patient at the predetermined location. The balloon catheter of the present disclosure; including the balloon as described herein, can be positioned over the guidewire and the balloon catheter can be advanced so as to position the balloon at or adjacent the predetermined location.

As one skilled in the art will appreciate, the balloon catheter can be positioned in the predetermined position in various ways, as described herein. In one embodiment, the balloon of the present disclosure can be deployed and placed in any number of intravascular locations. For example, the balloon catheter can be deployed and placed within a major artery of a patient. For the various embodiments, major arteries include, but are not limited to, the aorta. In addition, balloon catheters of the present disclosure can be deployed and placed within other intravascular positions of the heart and or other organs or tissues, such as the kidneys and/or cerebrovascular for help to condition the organ and/or tissue for a subsequent ischemic event or reperfusion. Other locations are also possible.

For the various embodiments, the method includes inflating the balloon of the balloon catheter to an expanded state. The balloon can be inflated by an inflation device releasably coupled to the balloon catheter (e.g., to the inflation port) from which a fluid (e.g., saline) can be transferred under pressure through the inflation lumen to inflate the balloon.

For the various embodiments, the method includes regulating a flow of the fluid past the balloon in the expanded state with a fluid control valve integrated with the balloon catheter at an adjustable rate greater than a non-flow rate and less than a full-flow rate of the fluid control valve. As discussed herein, the flow of the fluid past the balloon can be regulated by a valve member that moves relative to inlet openings on a perfusion lumen.

As discussed herein, the flow of the fluid past the balloon can also be regulated by a valve member that includes a plug positioned adjacent the inlet openings. For the various embodiments, the profile of the plug allows for the regulation of the flow of the fluid past the balloon at the adjustable rate greater than the no-flow rate and less than the full-flow rate.

For the various embodiments, regulation of the fluid flow may also be controlled by a valve member that can include a first end, a second end, and surface defining a lumen that extends from the first end to the second end, where the second end of the lumen is in fluid communication with the perfusion lumen. As discussed herein, the flow rate of the fluid past the balloon may be adjusted as the amount of the lumen of the second end of the valve member that is in fluid communication with the inlet openings changes.

Additionally, for the various embodiments, the fluid flow may also be controlled by introducing a fluid through a lumen of the valve member. For the various embodiments, regulating the flow of the fluid can include changing the inflation pressure of an inner balloon and outer balloon. The change in pressure changes the cross-sectional area of the tubular passage. Changing the cross-sectional area of the tubular passage allows the fluid flow to be regulated past the balloon at the adjustable rate greater than the no-flow rate and less then the full-flow rate of the fluid control valve.

For the various embodiments, the method can include monitoring a flow rate of the fluid with the balloon catheter. The monitoring of the flow rate of the fluid can be done by using a flow sensor, as discussed herein.

While the present disclosure has been shown and described in detail above, it will be clear to the person skilled in the art that changes and modifications may be made without departing from the spirit and scope of the disclosure. As such, that which is set forth in the foregoing description and accompanying drawings is offered by way of illustration only and not as a limitation. The actual scope of the disclosure is intended to be defined by the following claims, along with the full range of equivalents to which such claims are entitled. In addition, one of ordinary skill in the art will appreciate upon reading and understanding this disclosure that other variations for the disclosure described herein can be included within the scope of the present disclosure.

In the foregoing Detailed Description, various features are grouped together in several embodiments for the purpose of streamlining the disclosure. This method of disclosure is not to be interpreted as reflecting an intention that the embodiments of the disclosure require more features than are expressly recited in each claim. Rather, as the following claims reflect, inventive subject matter lies in less than all features of a single disclosed embodiment. Thus, the following claims are hereby incorporated into the Detailed Description, with each claim standing on its own as a separate embodiment.

What is claimed is:

1. A balloon catheter, comprising:
an elongate body having an inflation lumen;
a balloon coupled to the elongate body and in fluid communication with the inflation lumen; and
a fluid control valve integrated with the balloon catheter, where the fluid control valve regulates a flow of a fluid past the balloon in an expanded state at an adjustable rate greater than a no-flow rate and less than a full-flow rate.

2. The balloon catheter of claim 1, where the fluid control valve has a first surface defining an inlet opening through the elongate body into a perfusion lumen that extends longitudinally past the balloon through the elongate body to a second surface defining an outlet opening through the elongate body; and
a valve member that moves relative the inlet opening to regulate the flow of the fluid through the fluid control valve.

3. The balloon catheter of claim 2, where the valve member has a profile that allows for the flow of the fluid through the inlet opening and past the balloon at the adjustable rate greater than the no-flow rate and less than the full-flow rate.

4. The balloon catheter of claim 3, where the valve member includes a plug adjacent the inlet opening, where the valve member rotates the plug relative the inlet opening to regulate the flow of the fluid past the balloon at the adjustable rate greater than the no-flow rate and less than the full-flow rate.

5. The balloon catheter of claim 3, where the valve member includes a first end, a second end, and a surface defining a lumen that extends from the first end to the second end, where the second end of the lumen is in fluid communication with the perfusion lumen.

6. The balloon catheter of claim 2, where the valve member includes an electrode for conducting an electrical potential.

7. The balloon catheter of claim 2, where the perfusion lumen has a flow sensor to monitor the flow of the fluid past the balloon.

8. The balloon catheter of claim 1, where the fluid control valve includes a surface that defines a tubular passage longitudinally passing through the balloon, where changes in inflation pressure for the balloon in the expanded state regulate the flow of the fluid through the tubular passage past the balloon at the adjustable rate greater than the no-flow rate and less than the full-flow rate.

9. The balloon catheter of claim 8, where the balloon includes an outer balloon and an inner balloon concentrically arranged with and in fluid communication with the outer balloon, where the tubular passage past the balloon is between the outer balloon and the inner balloon so that changes in inflation pressure of the balloon change a cross-sectional area of the tubular passage to regulate the flow of the fluid past the balloon.

10. The method of forming a balloon catheter, comprising:
providing an elongate body having an inflation lumen;
coupling a balloon to the elongate body to be in fluid communication with the inflation lumen; and
integrating a fluid control valve with the balloon catheter, where the fluid control valve can regulate a flow of a fluid past the balloon in an expanded state at an adjustable rate greater than a no-flow rate and less than a full-flow rate.

11. The method of claim 10, where integrating the fluid control valve with the balloon catheter includes providing a perfusion lumen through at least a portion of the elongate body;
forming an inlet opening through the elongate body adjacent a first end of the balloon to connect with the perfusion lumen that extends past the balloon;
forming an outlet opening for the perfusion lumen through the elongate body adjacent a second end of the balloon; and
providing a valve member for the fluid control valve, where the valve member moves at least partially within the perfusion lumen and relative the inlet opening to regulate the flow of the fluid through the control valve.

12. The method of claim 11, including forming a lumen in the valve member, where the lumen can pass a liquid into the perfusion lumen of the elongate body.

13. The method of claim 11, including providing an electrode on the valve member, where the electrode can conduct an electrical potential.

14. The method of claim 10, where integrating the fluid control valve with the balloon catheter includes forming a tubular passage longitudinally past the balloon, where inflation pressure of the balloon regulates a flow of the fluid past the balloon in an expanded state at an adjustable rate greater than a no-flow rate and less than a full-flow rate.

15. The method of claim 14, where forming a tubular passage includes forming an outer balloon and an inner balloon concentrically arranged with and in fluid communication with the outer balloon; and
positioning the tubular passage past the balloon between the outer balloon and the inner balloon so that changes in inflation pressure of the balloon change a cross-sectional area of the tubular passage to regulate the flow of the fluid past the balloon.

16. A method, comprising:
positioning a balloon of a balloon catheter at a predetermined vascular location;
inflating the balloon of the balloon catheter to an expanded state; and
regulating a flow of a fluid past the balloon in the expanded state with a fluid control valve integrated with the balloon catheter at an adjustable rate greater than a no-flow rate and less than a full-flow rate.

17. The method of claim 16, where regulating the flow of the fluid includes moving a valve member of the fluid control valve to regulate the flow of the fluid.

18. The method of claim 17, including introducing a fluid through a lumen of the valve member.

19. The method of claim 16, where regulating the flow of the fluid includes increasing an inflation pressure for the balloon in the expanded state to regulate the flow of the fluid past the balloon at the adjustable rate greater than the no-flow rate and less than the full-flow rate.

20. The method of claim 16, including monitoring a flow rate of the fluid with the balloon catheter.
